SEQUENCE LISTING (29 sequences)

SEQ ID NO: 1 - DNA, 57 bp, Homo sapien
aagtagtgtgtgcccgtctgttactctggtaactagagatccctcagaggcacacac

SEQ ID NO: 2 - DNA, 47 bp, Homo sapien
aaccctggtgtctagagatccctcagatcacttagactgacaccagg

SEQ ID NO: 3 - DNA, 42 bp, Homo sapien
aatcttcaacgaggaatgcctagtagcgcaagtcaggcattc

SEQ ID NO: 4 - DNA, 38 bp, Homo sapien
cgtcagaggtgaaattcttagaccgcaccacacctctg

SEQ ID NO: 5 - DNA, 39 bp, Homo sapien
cgggctgtcaatatgctaaaacgcggaatgccgacagcc

SEQ ID NO: 6 - DNA, 41 bp, Homo sapien
atcgttcgttgagcgattagcagagaactgaccaacgaacg

SEQ ID NO: 7 - DNA, 40 bp, Homo sapien
agaggcaaccctgcactgttatggggcctacctggttgcc

SEQ ID NO: 8 - DNA, 35 bp, Homo sapien
acagcatattgacgctgggaaagaccagagcgtca

SEQ ID NO: 9 - DNA, 44 bp, Homo sapien
actctcattccaatggaaccttgttcaagttcaaaccattggaa

SEQ ID NO: 10 - DNA, 42 bp, Homo sapien
caccagcaccatgcaactttttcacctctgcctacatggtgc

SEQ ID NO: 11 - DNA, 58 bp, Homo sapien
atttgggcgtgcccccgcctagccgagtagtgttgggtcgcgaaaggccttgcacgcc

SEQ ID NO: 12 - DNA, 34 bp, Homo sapien
ttgtaccaacgcggtgccaatgcaagtggccgcg

SEQ ID NO: 13 - DNA, 34 bp, Homo sapien; identical to SEQ ID NO: 12

SEQ ID NO: 14 - DNA, 34 bp, Homo sapien; identical to SEQ ID NO: 12

SEQ ID NO: 15 - DNA, 56 bp, Homo sapien
aagtagtgtgtgcccgtctgttactctggtaactagagatccctcagaaccagagt

SEQ ID NO: 16 - DNA, 55 bp, Homo sapien
aagtagtgtgtgcccgtctgttactctggtaactagagatccctcagaccagagt

SEQ ID NO: 17 - DNA, 56 bp, Homo sapien
cccgtctgttgtgtgactctggtaactagagatccctcagactatttctagttacc

SEQ ID NO: 18 - DNA, 46 bp, Homo sapien
aaccctggtgtctagagatccctcagatcacttagactgatctgag

SEQ ID NO: 19 - DNA, 47 bp, Homo sapien
aaccctggtgtctagagatccctcagatcacttagactgaggatctc

SEQ ID NO: 20 - DNA, 41 bp, Homo sapien
aagactgctagccgagtagtgttgggtcgcgaaaccaacac

SEQ ID NO: 21 - DNA, 40 bp, Homo sapien
cgtgcccccgcaagactgctagccgagtagtgttgctagc

SEQ ID NO: 22 - DNA, 59 bp, Homo sapien
atttgggcgtgcccccgcctagccgagtagtgttgggtcgcgaaaggcctttactcggc

SEQ ID NO: 23 - DNA, 57 bp, Homo sapien
atttgggcgtgcccccgcctagccgagtagtgttgggtcgcgaaaggccttcgaccc

SEQ ID NO: 24 - DNA, 37 bp, Homo sapien
atttgggcgtgcccccgcaagactgctagccagcagt

SEQ ID NO: 25 - DNA, 37 bp, Homo sapien
atttgggcgtgcccccgcaagactgctagccgcagtc

SEQ ID NO: 26 - DNA, 38 bp, Homo sapien
atttgggcgtgcccccgcaagactgctagcccagtctt

SEQ ID NO: 27 - DNA, 42 bp, Homo sapien
tagcatggagctgtaggagtctaaattggggacttagactcc

SEQ ID NO: 28 - DNA, 39 bp, Homo sapien
tagcatggagctgtaggagtctaaattggggaccccaat

SEQ ID NO: 29 - DNA, 40 bp, Homo sapien
agcatggagctgtaggagtctaaattggggattagactcc

(19) United States
(12) Patent Application Publication
(10) Pub. No.: US 2017/0253878 A1
(43) Pub. Date: Sep. 7, 2017

(54) RAPID OLIGO PROBES

(71) Applicant: Co-Diagnostics, Inc., Sandy, UT (US)

(72) Inventor: Brent C. Satterfield, Chandler, AZ (US)

(21) Appl. No.: 15/280,304

(22) Filed: Sep. 29, 2016

Related U.S. Application Data
(63) Continuation of application No. 13/223,787, filed on Sep. 1, 2011, now abandoned.
Application No. 13/223,787 is a continuation of application No. PCT/US2010/025961, filed on Mar. 2, 2010.
(60) Provisional application No. 61/156,797, filed on Mar. 2, 2009.

(51) Int. Cl. C12N 15/115; C12Q 1/68

(52) U.S. Cl. CPC C12N 15/115; C12N 2310/16; C12N 2310/3517; C12N 2320/10; C12Q 1/6844; C12Q 2561/113; C12Q 2525/301

(57) ABSTRACT

Disclosed are methods, rapid probes, and kits for general purpose nucleic acid detection.

### CROSS-REFERENCE TO RELATED APPLICATIONS

This application is a continuation of and claims priority to PCT Application No. PCT/US2010/025961, filed on Mar. 2, 2010, entitled RAPID OLIGO PROBES, published as WO 2010/101947; and U.S. Provisional Application No. 61/156,797, filed on Mar. 2, 2009, also entitled RAPID OLIGO PROBES; each of which is incorporated herein by reference in its entirety.

### FIELD

The present technology is related to methods, rapid probes, and kits for general purpose nucleic acid detection.

### SEQUENCE LISTING

The present application is being filed along with a Sequence Listing in electronic format. The Sequence Listing is provided as a file entitled CDIAG.004C1.TXT, created Sep. 1, 2011 which is 4.05 Kb in size. Tue information in the electronic format of the Sequence Listing is incorporated herein by reference in its entirety.

### BACKGROUND

Labeled nucleic acid detection probes have been made to specifically detect a target analyte. Probe technologies include TAQMAN® probes (Life Technologies, Carlsbad, Calif.), TAQMAN® MGB probes (Life Technologies, Carlsbad, Calif.), MGB ECLIPSE™ probes (Nanogen, San Diego, Calif.), Molecular Beacons, FRET probes, Simple Probes, SCORPION™ primers (DxS Ltd., Manchester, UK) and AMPLIFLUOR® primers (Millipore, Billerica, Mass.).

Several of these probes, including Molecular Beacons, utilize a change in secondary structure to generate an increase in fluorescent signal, indicating the presence of the target nucleic acid sequence. However, the secondary structure can make probes sluggish or slow to react.

TENTACLE PROBES™ (Arcxis Biotechnologies, Pleasanton, Calif.) use cooperativity to increase the rate of reaction. Binding first occurs on a capture probe, which holds the target sequence in close proximity to the detection probe, allowing for a kinetically enhanced reaction. While this idea has been shown to improve the kinetics by as much as 200 fold over molecular beacons, the probe requires very complicated mathematics to construct correctly and the internal probe modifications drastically increase the synthesis costs. There is still a need for a relatively simple, low-cost probe with rapid kinetics.

### SUMMARY

The methods, probes, kits and other materials described herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the methods, probes, kits and other materials disclosed herein provide advantages over other known methods and materials.

Some embodiments relate to methods, rapid probes, and kits for detecting a target analyte in a specific manner. Probes may include, for example, a single strand of nucleic acids with labels at the 3′ and 5′ ends. Preferably, in some aspects, the probes can be single oligonucleotide strands. In some embodiments, a first sequence is complementary to a region internal to the 3′ or 5′ end of the probe. This first sequence enables the formation of a hairpin structure, bringing the two labels into close proximity. However, a third sequence of single-stranded nucleic acids extending beyond the hairpin structure allows for uninhibited hybridization with the target nucleic acid. Following seed nucleation, the rest of the probe hybridizes to the target like a zipper, disrupting the hairpin structure and causing a detectable change in signal. This method increases reaction kinetics, while maintaining a simple probe design and low-cost synthesis.

Various detection methods require contacting a probe with a target. Probes may be used in a variety of tests, including general hybridization assays and amplification assays.

In one embodiment, a probe for detecting the presence or absence of a target analyte in a sample is provided, comprising: a first sequence complementary to a region internal to the 3′ or 5′ end of the probe; a second sequence forming a hairpin or stem-loop structure when the first sequence is hybridized to the region internal to the 3′ or 5′ end of the probe; and a third sequence complementary to the target analyte, where the third sequence extends beyond the hairpin or stem-loop structure when the first sequence is hybridized with the region internal to the 3′ or 5′ end of the probe. In one aspect of this embodiment, the first, second, and third sequences are part of a single nucleic acid sequence. In another aspect of this embodiment, the probe comprises DNA. In a further aspect of this embodiment, the probe comprises RNA. In a further aspect of this embodiment, the first sequence comprises at least one nucleotide complementary to a variant analyte. In a further aspect of this embodiment, the probe is between about 10 nucleotides and about 70 nucleotides in length. In a further aspect of this embodiment, the probe is between about 20 nucleotides and about 50 nucleotides in length. In a further aspect of this embodiment, the probe is between about 30 nucleotides and about 40 nucleotides in length. In a further aspect of this embodiment, the third sequence is between about one nucleotide and about 40 nucleotides in length. In a further aspect of this embodiment, the third sequence is between about three nucleotides and about 20 nucleotides in length. In a further aspect of this embodiment, the third sequence is between about three nucleotides and about ten nucleotides in length. In a further aspect of this embodiment, the probe further comprises at least one fluorescent label affixed to the 3′ or 5′ region of the prob. In a further aspect of this embodiment, the probe further comprises a fluorescence quencher affixed to a 3′ or 5′ region of the probe that does not comprise a fluorescent label. In a further aspect of this embodiment, the probe has a sequence selected from the group consisting of SEQ ID NOs: 1 to 29.

In another embodiment, a kit comprising any of the probes described herein and a set of instructions for use of the probe is provided.

In a further embodiment, an assay for detecting the presence or absence of a target analyte in a sample, comprising a first sequence complementary to a region internal to the 3′ or 5′ end of the probe; a second sequence forming a hairpin or stem-loop structure when the first sequence is hybridized to the region internal to the 3′ or 5′ end of the probe; and a third sequence complementary to the target analyte, where the third sequence extends beyond the hairpin or stem-loop structure when the first sequence is hybridized with the region internal to the 3′ or 5′ end of the probe; and detecting the presence or absence of the target analyte in the sample. In one aspect of this embodiment, the detecting occurs in conjunction with nuclease cleavage of the probe. In another aspect of this embodiment, the detecting comprises detecting a change in secondary structure of the probe. In a further aspect of this embodiment, the detecting occurs in conjunction with an amplification reaction. In a further aspect of this embodiment, the detecting comprises detecting an interaction between a molecular energy transfer pair. In a further aspect of this embodiment, the detecting comprises detecting an interaction between an enzyme-inhibitor pair. In a further aspect of this embodiment, the assay further comprises contacting the sample with a second target specific probe. In a further aspect of this embodiment, the target analyte is a variant analyte. In a further aspect of this embodiment, the variant analyte comprises a single nucleotide polymorphism (SNP).

In a further embodiment, an isolated nucleic acid is provided, comprising: (a) a nucleic acid sequence selected from the group consisting of SEQ ID NOs: 1 to 29 and sequences complementary thereto; (b) a nucleic acid sequence having at least 80% identity to a nucleic acid sequence selected from the group consisting of SEQ ID NOs: 1 to 29 and sequences complementary thereto; (c) a nucleic acid sequence selected from the group consisting of SEQ ID NOs: 1 to 29 and sequences complementary thereto, where the nucleic acid sequence comprises about 1 to about 20 nucleotide analog substitutions or non-naturally occurring nucleotide substitutions; (d) a nucleic acid sequence having at least 10 consecutive nucleotides from a nucleic acid sequence selected from the group consisting of SEQ ID NOs: 1 to 29 and sequences complementary thereto; or (e) a sequence complementary to any of (a)-(d). In some aspects of part (b) the sequences can have at least 85%, 95%, 96%, 97%, 98% or 99% identity to a nucleic acid sequence of SEQ ID NOs. 1-29.

In a further embodiment, an isolated nucleotide sequence having the sequence of any of SEQ ID NOs 1 to 29 is provided.

In a further embodiment, a probe for detecting the presence or absence of a target nucleotide in a sample is provided, where the length of the probe is between about 10 and about 70 nucleotides; where the melting temperature of the probe-target nucleotide complex is at least about 15° C. above the reaction temperature for a binding reaction for the probe with the target nucleotide; where the melting temperature of the probe-target nucleotide complex is at least about 5° C. above the melting temperature of a primer-target complex for a polymerase chain reaction for the target nucleotide; where a single-stranded portion of the probe extends beyond a hairpin or stem-loop structure when a 5′ or 3′ region of the probe is hybridized to an internal portion of the probe; and where the melting temperature for the single-stranded portion of the probe extending beyond the hairpin or stem-loop structure is at least about 7° C. above the reaction temperature for the polymerase chain reaction.

In a further embodiment, a method of designing a probe to detect the presence or absence of a target analyte in a sample is provided, comprising: identifying a sequence of interest; and designing a probe as described herein to target the sequence of interest.

In a further embodiment, a probe for detecting the presence or absence of a target nucleotide in a sample is provided, where the length of the probe is between about 10 and about 70 nucleotides; where the melting temperature of the probe-target nucleotide complex is at least about 10° C.-20° C., preferably about 15° C., above the reaction temperature for a polymerase chain reaction for the target nucleotide; where the melting temperature of the probe-target nucleotide complex is at least about 3° C.-8° C., preferably about 5° C., above the melting temperature of a primer-target complex for a polymerase chain reaction for the target nucleotide; where a single-stranded portion of the probe extends beyond a hairpin or stem-loop structure when a 5′ or 3′ region of the probe is hybridized to an internal portion of the probe; and where the melting temperature for the single-stranded portion of the probe extending beyond the hairpin or stem-loop structure is at least about 7° C., preferably not more than 10° C., above the reaction temperature for the polymerase chain reaction. In one aspect of this embodiment, the probe further comprises a fluorophore and a quencher.

### BRIEF DESCRIPTION OF THE DRAWINGS

The foregoing and other features of the present disclosure will become more fully apparent from the following description and appended claims, taken in conjunction with the accompanying drawings. Understanding that the drawings depict only several embodiments in accordance with the disclosure and are not to be considered limiting of its scope, the disclosure will be described with additional specificity and detail through use of the accompanying drawings.

FIGS. 1A and 1B show detection of target by hybridization of rapid probe with target. In the presence of a target, the rapid probe anneals quickly with the target via the free end of the probe and undergoes a change in secondary structure, causing an increase in fluorescent intensity. FIG. 1A shows the first sequence causing formation of the hairpin structure is near the 3′ end of the probe. FIG. 1B shows the first sequence causing formation of the hairpin structure is near the 5′ end of the probe.

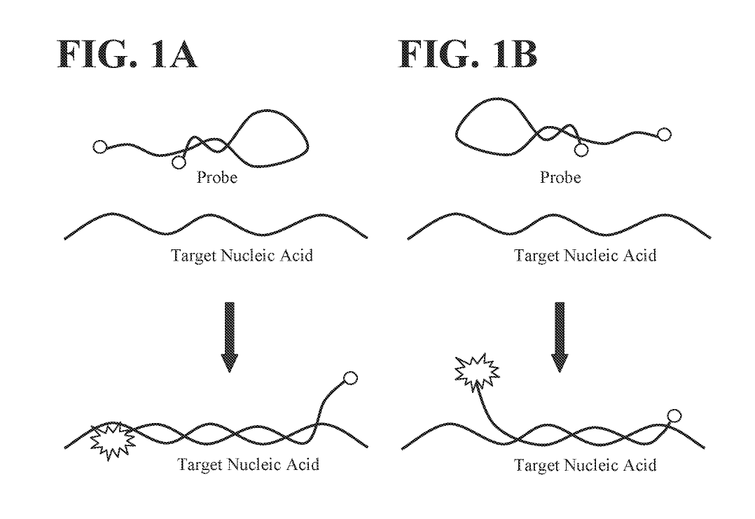

### DETAILED DESCRIPTION

The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

The present technology is in the field of methods, rapid probes, and kits for general purpose nucleic acid detection.

Some aspects relate, for example, to target-specific probes. For example, the probes can include a nucleic acid arm affixed to the end of the probe capable of hybridizing with a region internal to the probe, such that a portion of the probe extends beyond a hairpin that is formed. In some aspects, the probes can include single nucleic acid sequences.

To facilitate understanding of the disclosure that follows, a number of terms are defined below.

### Definitions

The term “amplicon” refers to a nucleic acid product generated in an amplification reaction.

The term “amplification” refers to the process in which “replication” is repeated at least once, and preferably more than once, in a cyclic process such that the number of copies of the nucleic acid sequence is increased in either a linear or logarithmic fashion.

The term “complementary strand” refers to a nucleic acid sequence strand which, when aligned with the nucleic acid sequence of one strand of the target nucleic acid, such that the 5′ end of the sequence is paired with the 3′ end of the other sequence in antiparallel association, forms a stable duplex. Complementarity need not be perfect. For example, stable duplexes can be formed with mismatched nucleotides.

The terms “detect,” “detection,” or “detecting the presence or absence of an analyte” refers to a process of providing qualitative or quantitative information about an analyte.

The term “label” refers to any atom or molecule that can be attached to or associated with a molecule for detection.

The term “nucleic acid” or “nucleotide” refers to a deoxyribonucleic acid (e.g., DNA, mtDNA, gDNA, or cDNA), ribonucleic acid (e.g., RNA or mRNA), nucleic acid analog, derivatives thereof, or any other variant of nucleic acids or nucleotides known in the art. There is no intended distinction between the length of a “nucleic acid,” “nucleotide,” “polynucleotide,” or “oligonucleotide.”

The term “peptide nucleic acid” or “PNA” refers to an analogue of DNA that has a backbone that comprises amino acids or derivatives or analogues thereof, rather than the sugar-phosphate backbone of nucleic acids (e.g., DNA and RNA). PNA mimics the behavior of a natural nucleic acid and binds complementary nucleic acid strands.

The term “primer” refers to an oligonucleotide that functions to initiate the nucleic acid replication or amplification process.

The term “probe” generally refers to a molecule having a desired affinity towards a target analyte. A probe can be an oligonucleotide in the broad sense, by which is meant that it can be DNA, RNA, or a mixture of DNA and RNA, and can include non-natural nucleotides and non-natural nucleotide linkages. A probe can also be a molecule other than an oligonucleotide, such as an amino acid, sugar, lectin, peptide, and the like. A probe functions in part by binding to a target analyte in a reaction mixture. Generally, a probe comprises a binding region that is capable of binding to an intended target region.

The terms “primer” and “probe” used herein can be used interchangeably and are not limited to oligonucleotides or nucleic acids, but rather encompass molecules that are analogs of nucleotides, as well as nucleosides. Nucleotides and polynucleotides, as used herein, shall be generic to polydeoxyribonucleotides (containing 2-deoxy-D-ribose), to polyribonucleotides (containing D-ribose), to any other type of polynucleotide which is an N- or C-glycoside of a purine or pyrimidine base, and to other polymers containing non-nucleotidic backbones, for example, polyamide (e.g., peptide nucleic acids (PNAs) and polymorpholino (commercially available from the Anti-Virals, Inc., Corvallis, Oreg. as Neugene™ polymers), and other synthetic sequence-specific nucleic acid polymers providing that the polymers contain nucleobases in a configuration which allows for base pairing and base stacking, such as is found in DNA and RNA.

Primers and/or probes can be provided in any suitable form, including suspension in liquid, in lyophilized form, or bound to a solid support.

The term “target” refers to the analyte to which a probe is intended to bind. In some embodiments, the target is the analyte which is being detected.

A probe can comprise an aptamer that can bind to its intended target. The term “aptamer” refers to a nucleic acid molecule that is capable of binding to a particular molecule of interest with high affinity and specificity (Tuerk and Gold, Science 249:505 (1990); Ellington and Szostak, Nature 346:818 (1990); which is incorporated herein by reference in its entirety). The binding of a ligand to an aptamer, which is typically RNA, changes the conformation of the aptamer and the nucleic acid within which the aptamer is located. The conformational change inhibits translation of an mRNA in which the aptamer is located, for example, or otherwise interferes with the normal activity of the nucleic acid. Aptamers may also be composed of DNA or may comprise non-natural nucleotides and nucleotide analogs. An aptamer will most typically have been obtained by in vitro selection for binding of a target molecule. However, in vivo selection of an aptamer is also possible.

The term “replication” refers to the process in which a complementary strand of a nucleic acid strand is synthesized by a polymerase enzyme. In a “primer directed” replication, this process generally requires a hydroxyl group (OH) at the 3′ end of (deoxy)ribose moiety of the terminal nucleotide of a duplexed “primer” to initiate replication.

The term “single nucleotide polymorphism” (SNP) refers to a single-base variation in the genetic code.

The term “variant” or “mutant” analyte refers to an analyte that is different than its wild type counterpart.

The term “wild type” as used herein refers to the typical form of an organism, strain, gene, or characteristic as it occurs in nature, as distinguished from mutant forms (e.g., forms that can result from selective breeding).

As used in this disclosure, the singular forms “a,” “an,” and “the” may refer to plural articles unless specifically stated otherwise. Thus, for example, references to a method of manufacturing, derivatizing, or treating “an analyte” may include a mixture of one or more analytes. Furthermore, the use of grammatical equivalents such as “nucleic acids,” “polynucleotides,” or “oligonucleotides” are not meant to imply differences among these terms unless specifically indicated.

### Analytes

Some embodiments relate to methods for detecting an analyte or a plurality of analytes. In some embodiments, methods can be used to analyze biological analytes. In some embodiments, methods can be used to analyze non-biological analytes. Suitable biological analytes may include, but are not limited to, proteins, peptides, nucleic acid sequences, peptide nucleic acids, antibodies, antigens, receptors, molecules, biological cells, microorganisms, cellular organelles, cell membrane fragments, bacteriophage, bacteriophage fragments, whole viruses, viral fragments, and small molecules such as lipids, carbohydrates, amino acids, drug substances, and molecules for biological screening and testing. An analyte can also refer to a complex of two or more molecules, for example, a ribosome with both RNA and protein elements or an enzyme with substrate attached.

In some embodiments, the target sequence of interest is a sequence from a disease causing agent, from a cancer cell or other neoplasm, a sequence indicative of a polymorphism in an animal, or a sequence indicative of a genetic abnormality in an animal.

In some embodiments, the sequence from a disease causing agent is selected from the group consisting of a bacterial sequence, a viral sequence, a fungal sequence, a plant sequence, a protist sequence, a micro animal sequence, and an archaea sequence. The sequence may also be from any other disease causing agent known to one of skill in the art.

In some embodiments, the sequence is a sequence from adenovirus, cytomegalovirus, Epstein-Barr virus, flavivirus, hantavirus, herpes simplex virus, influenza virus, varicella-zoster virus, hepatitis B virus, hepatitis C virus, hepatitis D virus, papilloma virus, parvovirus B19, polyomavirus BK, polyomavirus JC, rotavirus, measles virus, rubella virus, human immunodeficiency virus, human T cell leukemia virus, H. pylori, Streptococcus, Neisseria gonorrhoeae, Treponema pallidum, Chlamydia, Listeria, Salmonella, Legionella, Brucella, Coxiella, Rickettsia, Mycobacterium, Leishmania, Trypanosoma, Toxoplasma, and Plasmodium. The sequence may also be from any other infectious disease known to one of skill in the art.

In some embodiments, the cancer cell is selected from the group consisting of solid tumors or lymphomas such as leukemia, carcinoma, lymphoma, astrocytoma, sarcoma, glioma, retinoblastoma, melanoma, Wilm's tumor, bladder cancer, breast cancer, colon cancer, hepatocellular cancer, pancreatic cancer, prostate cancer, lung cancer, liver cancer, stomach cancer, cervical cancer, testicular cancer, renal cell cancer, or brain cancer. The cancer cell may also be any other cancer cell known to one of skill in the art.

In some embodiments, the sequence indicative of a genetic abnormality is selected from the group consisting of Angelman syndrome, Canavan disease, celiac disease, Charcot-Marie-Tooth disease, color blindness, cri du chat syndrome, cystic fibrosis, Down syndrome, Duchenne muscular dystrophy, familial hypercholesterolemia, hemophilia, Huntington disease, Marfan syndrome, neurofibromatosis, phenylketoneuria, polycystic kidney disease, Prader-Willi syndrome, sickle-cell disease, Tay-Sachs disease, and Turner syndrome. The genetic abnormality may also be any other genetic abnormality known to one of skill in the art.

In some embodiments, the analyte is able to specifically bind to at least a portion of the probe. The phrase “specifically bind(s)” or “bind(s) specifically” when referring to a detection probe refers to a detection probe that has intermediate or high binding affinity, exclusively or predominately, to a target molecule. The phrase “specifically binds to” refers to a binding reaction which is indicative of the presence of a target in the presence of a heterogeneous population of other biologics. Thus, under designated assay conditions, the specified binding region binds preferentially to a particular target and does not bind in a significant amount to other components present in a test sample. Specific binding to a target under such conditions can require a binding moiety that is selected for its specificity for a particular target. A variety of assay formats can be used to select binding regions that are specifically reactive with a particular analyte. Typically a specific or selective reaction will be at least twice the background signal or noise and more typically more than about 3 times, about 4 times, about 5 times, or about 10 times the background signal or noise.

Sources of analytes can be isolated from organisms and pathogens, such as viruses and bacteria, or from an individual or individuals, including, but not limited to, skin, plasma, serum, spinal fluid, lymph fluid, synovial fluid, urine, tears, blood cells, organs, tumors, and also samples of in vitro cell culture constituents, such as conditioned medium resulting from the growth of cells in cell culture medium, recombinant cells, and cell components. Analytes can also be from environmental samples such as air or water samples, or may be from forensic samples from biological or non-biological samples, including clothing, tools, publications, letters, furniture, etc. Additionally, analytes can also come from synthetic sources. The analytes can be provided in a sample that can be a crude sample, a partially purified or substantially purified sample, or a treated sample, where the sample can contain, for example, other natural components of biological samples, such as proteins, lipids, salts, nucleic acids, and carbohydrates.

A vast variety of modified nucleic acid analogs can also be used, including backbone modifications, sugar modifications, nitrogenous base modifications, or combinations thereof. The “backbone” of a natural nucleic acid is made up of one or more sugar-phosphodiester linkages. The backbone of a nucleic acid can also be made up of a variety of other linkages known in the art, including peptide bonds, also known as a peptide nucleic acid (Hyldig-Nielsen et al., PCT No. WO 95/32305; Egholm (1992) J. Am. Chem. Soc. 114:1895; Meier et al. (1992) Chem. Int. Ed. Engl. 31: 1008; Nielsen (1993) Nature 365:566; Carlsson et al. (1996) Nature 380:207); phosphorothioate linkages (Mag et al. (1991) Nucleic Acids Res. 19:1437; U.S. Pat. Nos. 5,644,048; 5,539,082; 5,773,571; 5,977,296, and 6,962,906); phosphorodithioate linkages (Briu et al. (1989) J. Am. Chem. Soc. 111:2321); phosphoramidate linkages (Beaucage et al. (1993) Tetrahedron 49(10):1925; Letsinger (1970) J. Org. Chem. 35:3800; Sprinzl et al. (1977) Eur. J. Biochem. 81:579; Letsinger et al. (1986) Nucleic Acids Res. 14:3487; Sawai et al. (1984) Chem. Lett. 805; Letsinger et al. (1988) J. Am. Chem. Soc. 110:4470; and Pauwels et al. (1986) Chemica Scripta 26:1419); methylphosphonate linkages; O-methylphophoroamidite linkages (Eckstein, Oligonucleotides and Analogues: A Practical Approach, Oxford University Press); or combinations thereof. Each of the above-listed references is incorporated herein by reference in its entirety. In some embodiments, any of the technology described in the listed references can be expressly excluded in whole or in part from the methods, primers, kits and other materials described herein.

Other suitable linkages include positive backbones (Denpcy et al. (1995) Proc. Natl. Acad. Sci. USA 92:6097); non-ionic backbones (U.S. Pat. Nos. 5,386,023, 5,637,684, 5,602,240, 5,216,141 and 4,469,863; Kiedrowski et al. (1991) Angew. Chem. Intl. Ed. English 30:423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research,” Ed. Y. S. Sanghui and P. Dan Cook; Mesmaeker et al. (1994), Bioorganic & Medicinal Chem. Lett. 4:395; Jeffs et al. (1994) J. Biomolecular NMR 34:17; Horn et al. (1996) Tetrahedron Lett. 37:743); and non-ribose backbones (U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook). Each of the above-listed references is incorporated herein by reference in its entirety. In some embodiments, any of the technology described in the listed references can be expressly excluded in whole or in part from the methods, primers, kits and other materials described herein.

Sugar moieties of a nucleic acid can be either ribose, deoxyribose, or similar compounds having known substitutions, such as 2′-O-methyl ribose, 2′-halide ribose substitutions (e.g., 2′-F), and carbocyclic sugars (Jenkins et al. (1995), Chem. Soc. Rev. pp169-176). The nitrogenous bases are conventional bases (A, G, C, T, U), known analogs thereof, such as inosine (I) (The Biochemistry of the Nucleic Acids 5-36, Adams et al., ed., 11th, 1992), known derivatives of purine or pyrimidine bases, such as N⁴-methyl deoxygaunosine, deaza- or aza-purines, deaza- or aza-pyrimidines, pyrimidine bases having substituent groups at the 5 or 6 position, purine bases having an altered or a replacement substituent at the 2, 6 or 8 positions, 2-amino-6-methylaminopurine, 0⁶-methylguanine, 4-thio-pyrimidines, 4-amino-pyrimidines, 4-dimethylhydrazine-pyrimidines, and 0⁴-alkyl-pyrimidines (Cook, PCT No. WO 93/13121), and “abasic” residues where the backbone includes no nitrogenous base for one or more residues of the polymer (Arnold et al., U.S. Pat. No. 5,585,481). Each of the above-listed references is incorporated herein by reference in its entirety. In some embodiments, any of the technology described in the listed references can be expressly excluded in whole or in part from the methods, primers, kits and other materials described herein.

In some embodiments, probes are designed to maximize reaction speed. In some embodiments, probes are designed to maximize signal generation. In some embodiments, probes are designed to maximize a combination of reaction speed and signal generation.

In some embodiments of the probe assay, probes can be designed for maximum specificity. In a preferred embodiment, the melting temperature of the 5′ overhang portion of the probe can be designed, for example, at between about 7° C. and about 10° C. over the reaction temperature. In some embodiments, the melting temperature of the hairpin structure can have a temperature of between about 7° C. and about 10° C. over the reaction temperature. In some embodiments in which probe assays operate based on base-pairing between nucleotides, preferred lengths for the 5′ or 3′ overhang portion of the probe (i.e., the single-stranded nucleic acid sequence extending beyond the hairpin) range between about 5 and about 25 bases, more preferably between about 10 and about 25 bases, and most preferably between about 15 and about 25 bases to achieve these melting temperatures ranges.

In some embodiments of the probe assay, large affinities may be desired for maximum sensitivity or to allow binding of variants. In some embodiments, melting temperatures for the probe can be designed, for example, to be between about 10° C. to about 50° C. over the reaction temperature, with the hairpin structure having a melting temperature of between about 7° C. and about 10° C. over the reaction temperature. In some embodiments targeting nucleic acids, total probe lengths can range, for example, preferably between about 10 and about 70 nucleotides, more preferably between about 20 and about 50 nucleotides, and most preferably between about 30 and about 40 nucleotides. In some embodiments, total probe lengths may range, for example, from between about 10 and 70 nucleotides, about 10 and 20 nucleotides, about 20 and 30 nucleotides, about 30 and 40 nucleotides, about 40 and 50 nucleotides, about 50 and 60 nucleotides, or about 60 and 70 nucleotides. In some embodiments, the total probe length is, is about, is at least, is at least about, is not more than, is not more than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides, for example.

In some embodiments, in order to maximize kinetics of a reaction, the length of the single-stranded nucleic acid sequence extending beyond the hairpin can be maximized, and the melting temperature of the hairpin structure does not exceed about 7° C. to about 10° C. over the reaction temperature. In some embodiments, in order to maximize a fluorescent signal, the length of the single-stranded nucleic acid sequence extending beyond the hairpin can be minimized. While extending the length of the single-stranded sequence beyond the hairpin is desirable, if it is extended too far, then the signal given upon hybridization will be reduced. Therefore, in some embodiments, the number of bases in the single-stranded nucleic acid sequence extending beyond the hairpin is between about 1 and about 40 bases, more preferably between about 4 and about 20 bases, and most preferably between about 10 and about 15 bases. In some embodiments, the number of bases in the single-stranded nucleic acid sequence ranges from between about 1 and 40 nucleotides, about 1 and 5 nucleotides, about 5 and 10 nucleotides, about 10 and 20 nucleotides, about 20 and 30 nucleotides, or about 30 and 40 nucleotides. In some embodiments, the number of bases in the single-stranded nucleic acid sequence is, is about, is at least, is at least about, is not more than, is not more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides.

### Signal Altering Moieties

The probes can contain signal altering moieties. In certain embodiments, the probe may contain a sequence complementary with another region of the probe. In the absence of a target, the probe exists predominantly in a closed stem-loop or hairpin conformation, with the sequence forming a duplex with the inner portion of the probe, and thus bringing the 5′ and 3′ ends in closer proximity for effective interaction, including, but not limited to, interaction between a molecular energy transfer pair or enzyme-inhibitor pair.

In general, upon binding to a target analyte, the interactions between the probe and the target analyte shifts the equilibrium predominantly towards to an open conformation. In this open conformation, the two ends are separated from each other, thus generating a change in detectable signal that can be used to detect or quantitate the target analyte. It will be understood that the labels can include or consist of multiple signal altering moieties if so desired.

A variety of signal altering groups are suitable for use in the probe. For example, signal altering moieties can include a wide range of energy donor and acceptor molecules to construct resonance energy transfer probes. Energy transfer can occur, for example, through fluorescence resonance energy transfer, bioluminescence energy transfer, or direct energy transfer. Fluorescence resonance energy transfer occurs when part of the energy of an excited donor is transferred to an acceptor fluorophore which re-emits light at another wavelength or, alternatively, to a quencher group that typically emits the energy as heat. There is a great deal of practical guidance available in the literature for selecting appropriate donor-acceptor pairs for particular probes, as exemplified by the following references: Pesce et al., Eds., Fluorescence Spectroscopy (Marcel Dekker, New York, 1971); White et al., Fluorescence Analysis: A Practical Approach (Marcel Dekker, New York, 1970); and the like. The literature also includes references providing exhaustive lists of fluorescent and chromogenic molecules and their relevant optical properties for choosing reporter-quencher pairs (see, for example, Berlman, Handbook of Fluorescence Spectra of Aromatic Molecules, 2nd Edition (Academic Press, New York, 1971); Griffiths, Colour and Constitution of Organic Molecules (Academic Press, New York, 1976); Bishop, Ed., Indicators (Pergamon Press, Oxford, 1972); Haugland, Handbook of Fluorescent Probes and Research Chemicals (Molecular Probes, Eugene, 1992) Pringsheim, Fluorescence and Phosphorescence (Interscience Publishers, New York, 1949); and the like. Further, there is extensive guidance in the literature for derivatizing acceptor and quencher molecules for covalent attachment via readily available reactive groups that can be added to a molecule. Many donor and acceptor molecules, in addition to synthesis techniques, are also readily available from many synthesis companies, such as Biosearch Technologies (Novato, Calif.). Each of the above-listed references is incorporated herein by reference in its entirety. In some embodiments, any of the technology described in the listed references can be expressly excluded in whole or in part from the methods, primers, kits and other materials described herein.

In certain embodiments, the first signal altering moiety is a fluorophore and the second signal altering moiety is a fluorescence quencher. In the absence of a target analyte, the probe is predominately in a closed conformation. Thus, the two signal altering moieties are close enough in space for effective molecular energy transfer, with the fluorescent signal of the fluorophore substantially suppressed by the fluorescence quencher. In the presence of a target analyte, the interactions between the target analyte and the probe change the conformation of the probe into an open state. Thus, the two signal altering moieties are far apart from each other in space and the fluorescent signal of the fluorophore is restored for detection.

In certain alternative embodiments, the first signal altering moiety and the second signal altering moieties are both fluorophores that emit a certain wavelength when in close proximity, and another when further apart.

Suitable fluorophores include, but are not limited to, coumarin, fluorescein (e.g., 5-carboxyfluorescein (5-FAM), 6-carboxyfluorescein (6-FAM), 2′,4′,1,4,-tetrachlorofluorescein (TET), 2′,4′,5′,7′,1,4-hexachlorofluorescein (HEX), and 2′,7′-dimethoxy-4′,5′-dichloro-6-carboxyfluorescein (JOE)), Lucifer yellow, rhodamine (e.g., tetramethyl-6-carboxyrhodamine (TAMRA), and tetrapropano-6-carboxyrhodamine (ROX)), 4,4-difluoro-5,7-dimethyl-4-bora-3a,4a-diaza-s-indacene (BODIPY), DABSYL, DABCYL, cyanine (e.g., Cy3, Cy5, and Cy7), eosine, Texas red, ROX, quantum dots, anthraquinone, nitrothiazole, and nitroimidazole compounds, Quasar and Cal-fluor dyes, and dansyl derivatives. Combination fluorophores such as fluorescein-rhodamine dimmers are also suitable (Lee et al. (1997) Nucleic Acids Res. 25:2816). Exemplary fluorophores of interest are further described in WO 01/42505 and WO 01/86001. Fluorophores can be chosen to absorb and emit in the visible spectrum or outside the visible spectrum, such as in the ultraviolet or infrared ranges. Each of the above-listed references is incorporated herein by reference in its entirety. In some embodiments, any of the technology described in the listed references can be expressly excluded in whole or in part from the methods, primers, kits and other materials described herein.

A fluorescence quencher is a moiety that, when placed very close to an excited fluorophore, causes there to be little or no fluorescence. Suitable quenchers described in the art include, but are not limited to, BLACK HOLE QUENCHERS™ (BHQ) (Biosearch Technologies, Novato, Calif.), rhodamine, tetramethyl rhodamine, pyrene butyrate, eosine nitrotyrosine, ethidium, fluorescein, Malachite green, Texas Red, and DABCYL and variants thereof, such as DABSYL, DABMI and Methyl Red. Fluorophores can also be used as quenchers, because they tend to quench fluorescence when touching certain other fluorophores. Suitable quenchers can be, for example, either chromophores such as DABCYL or malachite green, or fluorophores that do not fluoresce in the detection range when the detection oligonucleotide segment is in the open conformation. Gold nanoparticles, for example, are also suitable as fluorescent quenchers. In some embodiments, any of the technology described above can be expressly excluded in whole or in part from the methods, primers, kits and other materials described herein.

### Probes and Primers

In some embodiments, a specimen or sample can be contacted with a probe. In some embodiments, a specimen or sample can be contacted with a set of amplification primers. As used herein, the terms “primer” and “probe” can be used interchangeably, and can include, but are not limited to, oligonucleotides or nucleic acids. The terms “primer” and “probe” encompass molecules that are analogs of nucleotides, as well as nucleotides. Nucleotides and polynucleotides, as used herein, shall be generic to polydeoxyribonucleotides (containing 2-deoxy-D-ribose), to polyribonucleotides (containing D-ribose), to any other type of polynucleotide which is an N- or C-glycoside of a purine or pyrimidine base, and to other polymers containing non-nucleotidic backbones, for example, polyamide (e.g., peptide nucleic acids (PNAs)) and polymorpholino (commercially available from the Anti-Virals, Inc., Corvallis, Oreg., as NEUGENE™ polymers), and other synthetic sequence-specific nucleic acid polymers providing that the polymers contain nucleobases in a configuration which allows for base pairing and base stacking, such as is found in DNA and RNA.

The terms nucleotide and polynucleotide include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA. The terms also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides will also include modifications on the sugar moiety, e.g., wherein one or more of the hydroxyl groups are replaced with a halogen, an aliphatic group, or are functionalized as ethers, amines, or the like. Other modifications to nucleotides or polynucleotides involve rearranging, appending, substituting for, or otherwise altering functional groups on the purine or pyrimidine base which form hydrogen bonds to a respective complementary pyrimidine or purine. Tue resultant modified nucleotide or polynucleotide may form a base pair with other such modified nucleotidic units but not with A, T, C, G or U. For example, guanosine (2-amino-6-oxy-9-beta.-D-ribofuranosyl-purine) may be modified to form isoguanosine (2-oxy-6-amino-9-.beta.-D-ribofuranosyl-purine). Such modification results in a nucleoside base which will no longer effectively form a standard base pair with cytosine. However, modification of cytosine (1-.beta.-D-ribofuranosyl-2-oxy-4-amino-pyrimidine) to form isocytosine (1-D-ribofuranosyl-2-amino-4-oxy-pyrimidine) results in a modified nucleotide which will not effectively base pair with guanosine but will form a base pair with isoguanosine. Isocytosine is available from Sigma Chemical Co. (St. Louis, Mo.); isocytidine may be prepared by the method described by Switzer et al. (1993) Biochemistry 32: 10489-10496 and references cited therein; 2′-deoxy-5-methyl-isocytidine may be prepared by the method of Tor et al. (1993) J. Am. Chem. Soc. 115:4461-4467 and references cited therein; and isoguanine nucleotides may be prepared using the method described by Switzer et al. (1993), supra, and Mantsch et al. (1993) Biochem. 14:5593-5601, or by the method described U.S. Pat. No. 5,780,610 to Collins et al. Tue non-natural base pairs referred to as K and n., may be synthesized by the method described in Piccirilli et al. (1990) Nature 343:33-37 for the synthesis of 2,6-diaminopyrimidine and its complement (1-methylpyrazolo[4,3]-pyrimidine-5,7-(4H,6H)-dione. Other such modified nucleotidic units which form unique base pairs have been described in Leach et al. (1992) J. Am. Chem. Soc. 114:3675-3683 and Switzer et al., supra, or will be apparent to those of ordinary skill in the art. Each of the above-listed references is incorporated herein by reference in its entirety. In some embodiments, any of the technology described in the listed references can be expressly excluded in whole or in part from the methods, primers, kits and other materials described herein.

In some embodiments, a probe can include a detectable label. Labels of interest include directly detectable and indirectly detectable radioactive or non-radioactive labels such as fluorescent dyes. Directly detectable labels are those labels that provide a directly detectable signal without interaction with one or more additional chemical agents. Examples of directly detectable labels include fluorescent labels. Indirectly detectable labels are those labels which interact with one or more additional members to provide a detectable signal. In this latter embodiment, the label is a member of a signal producing system that includes two or more chemical agents that work together to provide the detectable signal. Examples of indirectly detectable labels include biotin or digoxigenin, which can be detected by a suitable antibody coupled to a fluorochrome or enzyme, such as alkaline phosphatase. In many preferred embodiments, the label is a directly detectable label. Directly detectable labels of particular interest include fluorescent labels. Fluorescent labels that find use in the subject invention include a fluorophore moiety. Specific fluorescent dyes of interest include: xanthene dyes, e.g., fluorescein and rhodamine dyes, such as fluorescein isothiocyanate (FITC), 2-[ethylamino)-3-(ethylimino)-2-7-dimethyl-3H-xanthen-9-yl]benzoic acid ethyl ester monohydrochloride (R6G)(emits a response radiation in the wavelength that ranges from about 500 to 560 nm), 1,1,3,3,3′,3′-Hexamethylindodicarbocyanine iodide (HIDC) (emits a response radiation in the wavelength that ranged from about 600 to 660 nm), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3 (emits a response radiation in the wavelength that ranges from about 540 to 580 nm), Cy5 (emits a response radiation in the wavelength that ranges from about 640 to 680 nm), etc; BODIPY dyes and quinoline dyes. Specific fluorophores of interest include: Pyrene, Coumarin, Diethylaminocoumarin, FAM, Fluorescein Chlorotriazinyl, Fluorescein, R 110, Eosin, JOE, R6G, HIDC, Tetramethylrhodamine, TAMRA, Lissamine, ROX, Napthofluorescein, Texas Red, Napthofluorescein, Cy3, and Cy5, and the like. In some embodiments, any of the technology described above in this paragraph can be expressly excluded in whole or in part from the methods, primers, kits and other materials described herein.

One of skill in the art will recognize that the primer and probe sequences disclosed herein can be modified to include additional nucleotides at the 5′ or the 3′ terminus. Likewise, in some embodiments, the primer and probe sequences can be modified by having nucleotides substituted within the sequence. It is recognized that the primer and probe sequences must contain enough complementarity to hybridize specifically to the respective target nucleic acid sequence.

### Amplification/Detection

Methods of amplification and/or detection can include any suitable method, including any method known to one of skill in the art. In some embodiments, a PCR is performed to amplify and/or detect sequences or products of interest. For example, quantitative PCR (“QPCR”) (also referred as “real-time PCR”) can provide quantitative measurements for sequences present in a biological sample. However, one of skill in the art of nucleic acid amplification knows the existence of other rapid amplification procedures, such as ligase chain reaction (LCR), transcription-based amplification systems (TAS), self-sustained sequence replication (3SR), nucleic acid sequence-based amplification (NASBA), strand displacement amplification (SDA), and branched DNA (bDNA) (Persing et al. (1993) Diagnostic Molecular Microbiology: Principles and Applications (American Society for Microbiology, Washington, D.C.; which is incorporated herein by reference in its entirety). The scope of this technology is not limited to the use of amplification by PCR, but rather includes the use of any rapid nucleic acid amplification methods or any other procedures that may be useful with the sequences of the embodiments described herein for the detection and/or quantification of sequences or products.

One of skill in the art will recognize in view of the instant disclosure that non-amplification methods can also be used for the generation and/or detection of target sequences or products. For example, methods in which a template is detected without amplification of a signal or the template (e.g., the method involving chemical detection of DNA binding as described in WO 2005/01122 (Adnavance Technologies, Inc., Vancouver, BC)) can be employed. In addition, methods in which a template is sequenced without amplification (e.g., the sequencing method as described in Eid et al., Science 2009 323(5910): 133-38) can be employed. The references described in this paragraph are incorporated herein by reference it their entireties. In some embodiments, any of the technology described above in this paragraph can be expressly excluded in whole or in part from the methods, primers, kits and other materials described herein.

### Sequences

Embodiments can have from about 50% to about 100% nucleic acid sequence identity to SEQ ID NOs: 1 to 29. That is, embodiments can have about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NOs: 1 to 29 or sequences complementary thereto.

Embodiments may include other fragments, modifications, derivatives, and variants of the nucleic acid sequences described herein. For example, nucleic acid embodiments of the technology can have from about 2 to about 59 consecutive nucleotides of a sequence of SEQ ID NOs: 1 to 29 or sequences complementary thereto. Some DNA fragments, for example, may include nucleic acids having less than or equal to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 consecutive nucleotides of a sequence of SEQ ID NOs: 1 to 29 or sequences complementary thereto. Preferably, the nucleic acid embodiments can include, for example, at least about 10, 11, 12, 13, 14, 15, 16, 17, 18, 19, or 20 consecutive nucleotides of a sequence of SEQ ID NOs: 1 to 29 or sequences complementary thereto. More preferably, the nucleic acid embodiments may include, for example, at least 10 consecutive nucleotides of a sequence of SEQ ID NOs: 1 to 29 or sequences complementary thereto. Embodiments also can include simplified sequences obtained by chemically modifying cytosines, e.g., as described in U.S. Patent Publication No. 20090042732, which is incorporated herein by reference in its entirety.

One of skill in the art will recognize that the primer and probe sequences disclosed herein can be modified to include additional nucleotides at the 5′ or the 3′ termini or both. For example, the length of the primer and probe sequences can be modified to include from about 1 to about 10 additional nucleotides or any number in between. Likewise, in some embodiments, the primer and probe sequences can be modified by having nucleotides substituted within the sequence. The substitutions can include substitutions of other naturally occurring nucleotides, artificial or non-naturally occurring nucleotides or any other chemical entity that may not technically be a nucleotide. For example, the sequences can include from about 1 to about 5 substitutions. In some embodiments, the number of substitutions may range, for example, from between about 1 and 35 nucleotides, about 1 and 5 nucleotides, about 5 and 10 nucleotides, about 10 and 15 nucleotides, about 15 and 20 nucleotides, about 20 and 25 nucleotides, about 25 and 30 nucleotides, or about 30 and 35 nucleotides. In some embodiments, the number of substitutions is, is about, is at least, is at least about, is not more than, is not more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides, for example. It is recognized that the modified primer and probe sequences preferably contain enough complementarity to hybridize specifically to the respective target nucleic acid sequence.

### Applications

The binding interaction of the probe with a target analyte can be monitored by the detection probe with an interactive label pair (the first and second signal altering moieties) as a donor-acceptor pair, such as a fluorophore-quencher pair. The detectable signal can be measured at one or more discrete time points, as in an end-point assay or continuously monitored in real-time as in a continuous assay. Detection of the signal can be performed in any appropriate way based, in part, upon the type of reporter or labeling molecule or employed as known in the art. In some embodiments, the signal can be compared against a control signal or standard curve. Non-limiting examples of existing apparatuses that may be used to monitor the reaction in real-time or take one or more single time point measurements include, Models 7300, 7500, and 7700 Real-Time PCR Systems (Applied Biosystems, Foster City, Calif.); the MYCYLER™ and!CYCLER® Thermal Cyclers (Bio-Rad, Hercules, Calif.); the MX3000P™ and MX4000® (Stratagene®, La Jolla, Calif.); the CHROMO 4™ Four-Color Real-Time System (MJ Research, Inc., Reno, Nev.); and the LIGHTCYCLER® 2.0 Instrument (Roche Applied Science, Indianapolis, Ind.).

The examples provided herein give those of ordinary skill in the art a disclosure and description of how to make and use some of the preferred embodiments, and are not intended to limit the scope of what the inventors regard as their invention. Modifications of the above-described modes for carrying out the some aspects of the technology that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference. In some embodiments, any of the technology described in the background or the references disclosed herein can be expressly excluded in whole or in part from the methods, primers, kits and other materials described herein.

### Examples

### Example 1: Rapid Probe Design

Rapid probes were designed with the following segments: a fluorophore at the 5′ end; a 5′ overhang complementary to the target sequence; an internal region; a 3′ stem complementary to a region of the probe with the highest GC content near the 5′ end; and a quencher at the 3′ end. The following general parameters were followed: a probe melting temperature (i.e., the melting temperature of the probe-target complex) at least 15° C. above the reaction temperature; a probe melting temperature at least 5° C. above the primer-target melting temperature; a 5′ overhang melting temperature at least 7° C. above the reaction temperature (and preferably not more than 10° C. above the reaction temperature); and a 3′ stem between 5 and 9 bases and complementary a region with the highest GC content near the 5′ end of the probe.

Non-limiting examples of rapid probes are shown in Table 1. Sequences denoted by lower case letters are complementary to an internal region near the 5′ end of the probe (i.e., the “first sequence,” as used herein). Sequences denoted by underscoring are complementary to the “first sequences.” Sequences located between sequences denoted by lower case letters and underscoring form hairpin loops (i.e., the “second sequence,” as used herein). Sequences located to the left of the underscored sequences are complementary to the target analytes and extend beyond the hairpin structures (i.e., the “third sequence,” as used herein).

### Example 2: PCR with Rapid Probes

Rapid probes corresponding to SEQ ID NOs: 1 to 14 from Table 1 were synthesized and run in a real-time PCR reaction on an AB StepOne™ real time PCR machine (Applied Biosystems, Foster City, Calif.). The concentration for each probe was 200 nM mixed in either Simplex DNA or Simplex RNA Master Mix (Cooperative Diagnostics, Greenwood, S.C., Cat# S1001 & S 1002). Primer concentrations were 500 nM. 5 uL of master mix was mixed with 5 uL of 200 fM template for each reaction. Thermal cycling conditions were 95° C. for 20 s followed by 45 cycles of 95° C. for 1 s and 55° C. for 20 s for DNA master mix; or 55° C. for 10 min, 95° C. for 20 s followed by 45 cycles of 95° C. for 1 s and 55° C. for 20 s for RNA master mix. The increase in fluorescence exhibited by each probe is shown as L′iRn in Table 1.

### Example 3: PCR with Rapid Probes

Rapid probes corresponding to SEQ ID NOs: 15 to 29 from Table 1 are synthesized and run in a real-time PCR reaction on an AB StepOne™ real time PCR machine (Applied Biosystems, Foster City, Calif.). The concentration for each probe is about 200 nM mixed in either Simplex DNA or Simplex RNA Master Mix (Cooperative Diagnostics, Greenwood, S.C., Cat# S1001 & S1002). Primer concentrations are 500 nM. 5 uL of master mix is mixed with 5 uL of 200 fM template for each reaction. Thermal cycling conditions are 95° C. for 20 s followed by 45 cycles of 95° C. for 1 s and 55° C. for 20 s for DNA master mix; or 55° C. for 10 min, 95° C. for 20 s followed by 45 cycles of 95° C. for 1 s and 55° C. for 20 s for RNA master mix.

TABLE 1

| SEQ | 5′ | | |
|---|---|---|---|
| ID | Fluoro- | | 3′ |
| NO: | phore | Sequence | Quencher |
| 1 | [FAM] | AAGTAGTGTGTGCCCGTCTGTTACTCTGGTAACTAGAGATCCCTCAGAggcacacac | [DABC] |
| 2 | [FAM] | AACCCTGGTGTCTAGAGATCCCTCAGATCACTTAGACTGAcaccagg | [DABC] |
| 3 | [FAM] | AATCTTCAACGAGGAATGCCTAGTAGCGCAAGTCAggcattc | [DABC] |
| 4 | [FAM] | CGTCAGAGGTGAAATTCTTAGACCGCACCAcacctctg | [DABC] |
| 5 | [FAM] | CGGGCTGTCAATATGCTAAAACGCGGAATGCCgacagcc | [DABC] |
| 6 | [FAM] | ATCGTTCGTTGAGCGATTAGCAGAGAACTGACCAacgaacg | [DABC] |
| 7 | [FAM] | AGAGGCAACCCTGCACTGTTATGGGGCCTACCTggttgcc | [DABC] |
| 8 | [FAM] | ACAGCATATTGACGCTGGGAAAGACCAGAgcgtca | [DABC] |
| 9 | [FAM] | ACTCTCATTCCAATGGAACCTTGTTCAAGTTCAAAccattggaa | [DABC] |
| 10 | [FAM] | CACCAGCACCATGCAACTTTTTCACCTCTGCCTAcatggtgc | [DABC] |
| 11 | [FAM] | ATTTGGGCGTGCCCCCGCCTAGCCGAGTAGTGTTGGGTCGCGAAAGGCCTTgcacgcc | [DABC] |
| 12 | [FAM] | TTGTACCAACGCGGTGCCAATGCAAGTGGccgcg | [BHQ] |
| 13 | [FAM] | TTGTACCAACGCGGTGCCAATGCAAGTGGccgcg | [BHQ] |
| 14 | [FAM] | TTGTACCAACGCGGTGCCAATGCAAGTGGccgcg | [BHQ] |
| 15 | [FAM] | AAGTAGTGTGTGCCCGTCTGTTACTCTGGTAACTAGAGATCCCTCAGAaccagagt | [DABC] |
| 16 | [FAM] | AAGTAGTGTGTGCCCGTCTGTTCTCTGGTAACTAGAGATCCCTCAGAccagagt | [DABC] |
| 17 | [FAM] | CCCGTCTGTTGTGTGACTCTGGTAACTAGAGATCCCTCAGACTATTTctagttacc | [DABC] |
| 18 | [FAM] | AACCCTGGTGTCTAGAGATCCCTCAGATCACTTAGACTGAtctgag | [DABC] |
| 19 | [FAM] | AACCCTGGTGTCTAGAGATCCCTCAGATCACTTAGACTGAggatctc | [DABC] |
| 20 | [FAM] | AAGACTGCTAGCCGAGTAGTGTTGGGTCGCGAAAccaacac | [DABC] |
| 21 | [FAM] | CGTGCCCCCGCAAGACTGCTAGCCGAGTAGTGTTgetage | [DABC] |
| 22 | [FAM] | ATTTGGGCGTGCCCCCGCCTAGCCGAGTAGTGTTGGGTCGCGAAAGGCCTTtactcggc | [DABC] |
| 23 | [FAM] | ATTTGGGCGTGCCCCCGCCTAGCCGAGTAGTGTTGGGTCGCGAAAGGCCTTcgaccc | [DABC] |
| 24 | [FAM] | ATTTGGGCGTGCCCCCGCAAGACTGCTAGCCagcagt | [DABC] |
| 25 | [FAM] | ATTTGGGCGTGCCCCCGCAAGACTGCTAGCCgcagtc | [DABC] |
| 26 | [FAM] | ATTTGGGCGTGCCCCCGCAAGACTGCTAGCCcagtctt | [DABC] |
| 27 | [FAM] | TAGCATGGAGCTGtAGGAGTCTAAATTGGGGACttagactcc | [DABC] |
| 28 | [FAM] | TAGCATGGAGCTGtAGGAGTCTAAATTGGGGACcccaat | [DABC] |
| 29 | [FAM] | AGCATGGAGCTGtAGGAGTCTAAATTGGGGAttagactcc | [DABC] |

| | | | | Length of | | | |
|---|---|---|---|---|---|---|---|
| SEQ | | 5′ | Length of | Target | | Total | |
| ID | Probe | Over-hang | 5′ | Recognition | Length of | Probe | |
| NO: | Tₘ | Tₘ | Overhang | Sequence | 3′ Stem | Length | Δ Rn |
| 1 | 75.6 | 66.2 | 5 | 48 | 9 | 57 | 35000 |
| 2 | 72.6 | 65.2 | 3 | 40 | 7 | 47 | 32000 |
| 3 | 761 | 64i | 13 | 35 | 7 | 42 | 13000 |
| 4 | 73.9 | 64.2 | 3 | 30 | 8 | 38 | 6000 |
| 5 | 74.7 | 63.6 | 2 | 32 | 7 | 39 | 2000 |
| 6 | 75 | 65.8 | 2 | 34 | 7 | 41 | 20000 |
| 7 | 78.5 | 64.7 | 3 | 33 | 7 | 40 | 57000 |
| 8 | 71 | 66.8 | 9 | 29 | 6 | 35 | 10000 |
| 9 | 70.8 | 64.8 | 7 | 35 | 9 | 44 | 10000 |
| 10 | 771 | 67.9 | 5 | 34 | 8 | 42 | 32500 |
| 11 | 83.3 | 65.1 | 5 | 51 | 7 | 58 | 32000 |
| 12 | 89.6 | 68.9 | 9 | 29 | 5 | 34 | 40000 |
| 13 | 89.6 | 68.9 | 9 | 29 | 5 | 34 | 50000 |
| 14 | 89.6 | 68.9 | 9 | 29 | 5 | 34 | 1500 |
| 15 | 75.6 | 63.5 | 21 | 48 | 8 | 56 | N/A |
| 16 | 75.6 | 63.7 | 22 | 48 | 7 | 55 | N/A |
| 17 | 76.2 | 60.4 | 20 | 47 | 9 | 56 | N/A |
| 18 | 76 | 62.7 | 21 | 40 | 6 | 46 | N/A |
| 19 | 76 | 64.7 | 14 | 40 | 7 | 47 | N/A |
| 20 | 77.8 | 65.6 | 18 | 34 | 7 | 41 | N/A |
| 21 | 81.5 | 61.5 | 17 | 34 | 6 | 40 | N/A |
| 22 | 84.4 | 64.6 | 21 | 51 | 8 | 59 | N/A |
| 23 | 84.4 | 66.3 | 34 | 51 | 6 | 57 | N/A |
| 24 | 81 | 64.3 | 21 | 31 | 6 | 37 | N/A |
| 25 | 81 | 64.1 | 20 | 31 | 6 | 37 | N/A |
| 26 | 81 | 65 | 18 | 32 | 6 | 38 | N/A |
| 27 | 72.4 | 62.2 | 15 | 33 | 9 | 42 | N/A |
| 28 | 72.4 | 63.2 | 24 | 33 | 6 | 39 | N/A |
| 29 | 72.8 | 62.7 | 14 | 31 | 9 | 40 | N/A |

What is claimed is:

1. An isolated nucleic acid comprising:
(a) a nucleic acid sequence selected from the group consisting of SEQ ID NOs: 1 to 29 and sequences complementary thereto;
(b) a nucleic acid sequence having at least 80% identity to a nucleic acid sequence selected from the group consisting of SEQ ID NOs: 1 to 29 and sequences complementary thereto;
(c) a nucleic acid sequence selected from the group consisting of SEQ ID NOs: 1 to 29 and sequences complementary thereto, wherein the nucleic acid sequence comprises about 1 to about 20 nucleotide analog substitutions or non-naturally occurring nucleotide substitutions;
(d) a nucleic acid sequence having at least 10 consecutive nucleotides from a nucleic acid sequence selected from the group consisting of SEQ ID NOs: 1 to 29 and sequences complementary thereto; or
(e) a sequence complementary to any of (a)-(d).

2. The isolated nucleic acid of claim 1, wherein the nucleic acid has a nucleotide sequence having the sequence of any of SEQ ID NOs 1 to 29.

3. A probe for detecting the presence or absence of a target nucleotide in a sample, wherein the length of the probe is between about 10 and about 70 nucleotides; wherein the melting temperature of the probe-target nucleotide complex is at least about 15° C. above the reaction temperature for a binding reaction for the probe with the target nucleotide; wherein the melting temperature of the probe-target nucleotide complex is at least about 5° C. above the melting temperature of a primer-target complex for a polymerase chain reaction for the target nucleotide; wherein a single-stranded portion of the probe extends beyond a hairpin or stem-loop structure when a 5′ or 3′ region of the probe is hybridized to an internal portion of the probe; and wherein the melting temperature for the single-stranded portion of the probe extending beyond the hairpin or stem-loop structure is at least about 7° C. above the reaction temperature for the polymerase chain reaction.

4. The probe of claim 3, further comprising a fluorophore and a quencher.

5. A probe for detecting the presence or absence of a target analyte in a sample, comprising:
a first sequence complementary to a region internal to the 3′ or 5′ end of the probe;
a second sequence forming a hairpin or stem-loop structure when the first sequence is hybridized to the region internal to the 3′ or 5′ end of the probe; and
a third sequence complementary to the target analyte, wherein the third sequence extends beyond the hairpin or stem-loop structure when the first sequence is hybridized with the region internal to the 3′ or 5′ end of the probe.

6. The probe of claim 5, wherein the first, second, and third sequences are part of a single nucleic acid sequence.

7. The probe of claim 5, further comprising at least one fluorescent label affixed to the 3′ or 5′ region of the probe.

8. The probe of claim 7, further comprising a fluorescence quencher affixed to a 3′ or 5′ region of the probe that does not comprise a fluorescent label.

9. The probe of claim 5, wherein the probe comprises DNA or RNA.

10. The probe of claim 5, wherein the first sequence comprises at least one nucleotide complementary to a variant analyte.

11. The probe of claim 10, wherein the probe is between about 10 nucleotides and about 70 nucleotides in length.

12. The probe of claim 5, wherein the third sequence is between about one nucleotide and about 40 nucleotides in length.

13. The probe claim 5, having a sequence selected from the group consisting of SEQ ID NOs: 1 to 29.

14. An assay for detecting the presence or absence of a target analyte in a sample, comprising:
obtaining a sample comprising a target analyte;
contacting the sample with a target specific probe, wherein the probe is a continuous nucleotide sequence comprising:
a first sequence complementary to a region internal to the 3′ or 5′ end of the probe;
a second sequence forming a hairpin or stem-loop structure when the first sequence is hybridized to the region internal to the 3′ or 5′ end of the probe; and
a third sequence complementary to the target analyte, wherein the third sequence extends beyond the hairpin or stem-loop structure when the first sequence is hybridized with the region internal to the 3′ or 5′ end of the probe; and
detecting the presence or absence of the target analyte in the sample.

15. The assay of claim 14, wherein the detecting occurs in conjunction with nuclease cleavage of the probe or in conjunction with an amplification reaction.

16. The assay of claim 14, wherein the detecting comprises detecting a change in secondary structure of the probe, detecting an interaction between a molecular energy transfer pair, or detecting an interaction between an enzyme-inhibitor pair.

17. The assay of claim 14, further comprising contacting the sample with a second target specific probe.

18. The assay of claim 17, wherein the target analyte is a variant analyte.

19. The assay of claim 18, wherein the variant analyte comprises a single nucleotide polymorphism (SNP).
